(19) United States
(12) Patent Application Publication
(10) Pub. No.: US 2008/0153827 A1
(43) Pub. Date: Jun. 26, 2008

(54) Method for the Treatment or Prevention of Cardiac Hypertrophy

(76) Inventors: Heimo Haikala, Espoo (FI); Petri Kaheinen, Helsinki (FI); Jouko Levijoki, Helsinki (FI); Piero Pollesello, Grankulla (FI); Eero Mervaala, Espoo (FI)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/596,065

(22) PCT Filed: May 12, 2005
(86) PCT No.: PCT/FI05/00219

§ 371 Date: Aug. 27, 2007

(30) Foreign Application Priority Data
May 12, 2004 (FI) 20040675

(51) Int. Cl. A61K 31/50; A61P 9/00

(52) U.S. Cl. 514/247

(57) ABSTRACT

A method for the treatment or prevention of cardiac hypertrophy or diastolic heart failure resulting from cardiac hypertrophy by administering levosimendan or its metabolite (II) or any of their pharmaceutically acceptable salts to a mammal in need of such treatment.

### TECHNICAL FIELD

The present invention relates to a method for the treatment or prevention of cardiac hypertrophy by administering levosimendan or its metabolite (II) or any of their pharmaceutically acceptable salts, to a mammal in need of such treatment.

### BACKGROUND OF THE INVENTION

Levosimendan, which is the (−)-enantiomer of [[4-(1,4,5,6-tetrahydro-4-methyl-6-oxo-3-pyridazinyl)phenyl]hydrazono]propanedinitrile, and the method for its preparation is described in EP 565546 B1. Levosimendan is potent in the treatment of heart failure and has significant calcium dependent binding to troponin. Levosimendan is represented by the formula:

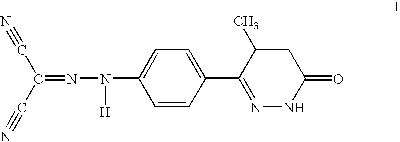

The hemodynamic effects of levosimendan in man are described in Sundberg, S. et al., Am. J. Cardiol., 1995; 75: 1061-1066 and in Lilleberg, J. et al., J. Cardiovasc. Pharmacol., 26(Suppl. 1), S63-S69, 1995. Pharmacokinetics of levosimendan in man after i.v. and oral dosing is described in Sandell, E.-P. et al., J. Cardiovasc. Pharmacol., 26(Suppl. 1), S57-S62, 1995. Clinical studies have confirmed the beneficial effects of levosimendan in heart failure patients.

Recently it has been found that levosimendan has an active metabolite (R)-N-[4-(1,4,5,6-tetrahydro-4-methyl-6-oxo-3-pyridazinyl)phenyl]acetamide (II) which is present in human following administration of levosimendan. The effects of (II) are similar to levosimendan. The use of (II) for increasing calcium sensitivity of contractile proteins in the cardiac muscle has been described in WO 99/66932.

Cardiac hypertrophy is an adaptive response of the heart to hemodynamic overload such as systemic hypertension. It is defined by an enlargement of the heart due in part to an increase in the size of the myocytes. Cardiac hypertrophy can be measured by various parameters including left ventricular mass:body weight ratio, changes in cardiomyocyte size, mass and organisation, changes in cardiac gene expression and fibroid deposition. Cardiac hypertrophy is typically confirmed by echo cardiography.

Mechanical stretch induced by hypertension is an initial factor in the development of cardiac hypertrophy. Sustained hypertension is known to result in cardiac hypertrophy. A characteristic of a ventricle that becomes hypertrophic as a result of chronic pressure overload is an impaired diastolic performance and increased chamber stiffness during diastole. A prolonged left ventricular relaxation has been detected in early essential hypertension.

Although the hypertrophic process can initially be compensatory, with severe long-standing overload the hypertrophied cells begin to deteriorate and die. Cardiac hypertrophy has been correlated with an increase in morbidity and mortality in cardiovascular diseases. Cardiac hypertrophy is also a risk factor for arrhythmia and sudden death.

Current medical management of cardiac hypertrophy includes the use of certain antihypertensive drugs such as calcium channel blockers, diuretics, beta-adrenergic blockers, angiotensin converting enzyme (ACE) inhibitors and angiotensin II receptor blockers. Although certain antihypertensive drugs have been shown to reduce left ventricular mass, treatment does not always result in improvement of diastolic function. Moreover, lowering of the elevated blood pressure to the normal level does not necessarily cause an improvement in cardiac hypertrophy. Indeed, despite of successful management of hypertension a substantial number (5-50%) of patients develop cardiac hypertrophy.

Despite currently available pharmaceutical agents, prevention and treatment of cardiac hypertrophy continue to present a therapeutic challenge. Thus, novel treatments for inhibiting the excessive formation of cardiac hypertrophy or reducing the hypertrophy would be highly desired.

### SUMMARY OF THE INVENTION

It has now been found that levosimendan and its active metabolite (II) attenuated significantly the experimentally induced cardiac hypertrophy in hypertensive rats even though the elevated blood pressure was not affected. Moreover, the effect was seen already at low plasma concentrations. The results indicate that the hypertrophy inhibiting action was independent of vasodilatation. Thus, the present invention provides a new method for controlling chronic cardiac hypertrophy. The method may also be useful for patients who develop cardiac hypertrophy despite controlled blood pressure.

Therefore, the present invention provides the use of levosimendan or its active metabolite (II) or any of their pharmaceutically acceptable salts in the manufacture of a medicament for the treatment or prevention of cardiac hypertrophy.

The present invention also provides the use of levosimendan or its active metabolite (II) or any of their pharmaceutically acceptable salts in the manufacture of a medicament for the treatment or prevention of diastolic heart failure resulting from cardiac hypertrophy.

The present invention also provides a method for the treatment or prevention of cardiac hypertrophy in a mammal, said method comprising administering to a mammal in need thereof an effective amount of levosimendan or its metabolite (II) or any of their pharmaceutically acceptable salts.

The present invention also provides a method for the treatment or prevention of diastolic heart failure resulting from cardiac hypertrophy in a mammal, said method comprising administering to a mammal in need thereof an effective amount of levosimendan or its metabolite (II) or any of their pharmaceutically acceptable salts.

### BRIEF DESCRIPTION OF THE DRAWINGS

FIG. 1 shows the ratio of the heart weight to the body weight of Dahl salt-sensitive rats on high salt diet treated with levosimendan at two different doses (Dahl HS+levo 1 and Dahl HS+levo 10) compared to that for untreated Dahl salt-sensitive rats on high salt (Dahl HS) diet and Dahl salt-sensitive rats on low salt (Dahl LS) diet.

FIG. 2 shows the ratio of myocardial SERCA2 expression to myocardial NCX expression in Dahl salt-sensitive rats on high salt diet treated with levosimendan at two different doses (Dahl HS+levo 1 and Dahl HS+levo 10) compared to that for untreated Dahl salt-sensitive rats on high salt (Dahl HS) diet and Dahl salt-sensitive rats on low salt (Dahl LS) diet.

FIG. 3 shows the mRNA amount of atrial natriuretic peptide (ANP) in Dahl salt-sensitive rats on high salt diet treated with levosimendan at two different doses (Dahl HS+levo 1 and Dahl HS+levo 10) compared to that for untreated Dahl salt-sensitive rats on high salt (Dahl HS) diet and Dahl salt-sensitive rats on low salt (Dahl LS) diet.

FIG. 4 shows interventricular septum (IVS) wall thickness (mm) of the heart in Dahl salt-sensitive rats on low salt diet (1), on high salt diet (2), on high salt diet treated with high dose levosimendan (3), on high salt diet treated with low dose levosimendan (4) and on high salt diet treated with active metabolite (II) of levosimendan (5).

### DETAILED DESCRIPTION OF THE INVENTION

As used herein the term “cardiac hypertrophy” means pathological enlargement of the heart due in part to an increase in the size or mass of the myocytes.

The term “diastolic heart failure” means a pathological state of diastolic dysfunction in which heart relaxation, in particular the filling of left ventricle, is impaired. In such diastolic dysfunction, the heart muscle fails to relax properly between beats. The increased stiffness of the heart during diastole generates excessive resistance of the heart chamber to refilling. In its simplest terms, diastolic dysfunction translates to the reduced ability of the heart to fill with blood. Traditional therapy, which is generally directed at improving systolic performance, is not applicable to treating diastolic dysfunction.

The method according to the invention relates to administering to a subject an amount of levosimendan or its active metabolite (II) effective to reduce, inhibit or prevent cardiac hypertrophy or formation of cardiac hypertrophy, particularly cardiac hypertrophy caused by pressure overload, in a mammal, including man. Preferably, the cardiac hypertrophy reducing effect is independent of lowering blood pressure in a patient. The pressure overload is typically systemic hypertension but can result also from other disease states such as valvular heart disease or aortic stenosis.

According to one preferred embodiment of the invention, the cardiac hypertrophy to be treated or prevented is hypertension-induced cardiac hypertrophy.

According to another embodiment of the invention, levosimendan or its metabolite (II) or any of their pharmaceutically acceptable salts is used in the treatment or prevention of cardiac hypertrophy independent of lowering elevated blood pressure.

According to another embodiment of the invention, levosimendan or its metabolite (II) or any of their pharmaceutically acceptable salts is used in the treatment or prevention of cardiac hypertrophy independent of inhibiting myocardial ischemia or arrhythmias.

The method according to the invention also relates to administering to a subject an amount of levosimendan or its active metabolite (II) effective to reduce, inhibit or prevent diastolic heart failure resulting from cardiac hypertrophy in a mammal, including man. Reducing cardiac hypertrophy is expected to decrease chamber stiffness and improve elastic properties of the myocardium, thereby improving the filling of left ventricle.

The administration of levosimendan or its active metabolite (II) can be enteral, e.g. oral or rectal; parenteral, e.g. intravenous; or transdermal or transmucosal.

The effective amount of levosimendan or its active metabolite (II) to be administered to a subject depends upon the condition to be treated or prevented, the route of administration, age, weight and the condition of the patient. Oral daily dose of levosimendan or its active metabolite (II) in man ranges generally from about 0.05 to about 10 mg. For the long-term treatment or prevention of cardiac hypertrophy in man, relatively low oral doses are generally preferred, e.g. an oral daily dose from about 0.05 to about 5 mg, preferably from about 0.1 to about 4 mg, more preferably from about 0.2 to about 3 mg.

Levosimendan can be administered by intravenous infusion using the infusion rate from about 0.01 to 5 μg/kg/min, more typically from about 0.02 to 3 μg/kg/min. The active metabolite (II) can be administered intravenously using an infusion rate, which is from about 0.001 to 1 μg/kg/min, preferably from about 0.005 to 0.5 μg/kg/min.

The active ingredient of the invention may be administered daily or several times a day or periodically, e.g. weekly or biweekly, depending on the patient's needs.

For the long-term treatment or prevention of cardiac hypertrophy, oral administration is preferred. Particularly preferred active ingredient is levosimendan or a pharmaceutically acceptable salt thereof.

Levosimendan or its active metabolite (II) is formulated into dosage forms suitable for the treatment or prevention of cardiac hypertrophy using the principles known in the art. It is given to a patient as such or preferably in combination with suitable pharmaceutical excipient in the form of tablets, granules, capsules, suppositories, emulsions, suspensions or solutions whereby the contents of the active compound in the formulation is from about 0.1 to 100% per weight. Choosing suitable ingredients for the composition is a routine for those of ordinary skill in the art. It is evident that suitable carriers, solvents, gel forming ingredients, dispersion forming ingredients, antioxidants, colours, sweeteners, wetting compounds, release controlling components and other ingredients normally used in this field of technology may be also used.

For oral administration in tablet form, suitable carriers and excipients include e.g. lactose, corn starch, magnesium stearate, calcium phosphate and talc. For oral administration in capsule form, useful carriers and excipients include e.g. lactose, corn starch, magnesium stearate and talc. For controlled release oral compositions release controlling components can be used. Typical release controlling components include hydrophilic gel forming polymers such as hydroxypropylmethyl cellulose, hydroxypropyl cellulose, carboxymethyl celluloses, alginic acid or a mixture thereof; vegetable fats and oils including vegetable solid oils such as hydrogenated soybean oil, hardened castor oil or castor seed oil (sold under trade name Cutina HR), cotton seed oil (sold under the trade names Sterotex or Lubritab) or a mixture thereof; fatty acid esters such as triglycerides of saturated fatty acids or their mixtures e.g. glyceryl tristearates, glyceryl tripalmitates, glyceryl trimyristates, glyceryl tribehenates (sold under the trade name Compritol) and glyceryl palmitostearic acid ester.

Tablets can be prepared by mixing the active ingredient with the carriers and excipients and compressing the powdery mixture into tablets. Capsules can be prepared by mixing the active ingredient with the carriers and excipients and placing the powdery mixture in capsules, e.g. hard gelatin capsules. Typically a tablet or a capsule comprises from about 0.05 to 10 mg, more typically from about 0.2 to 4 mg, of levosimendan or its active metabolite (II).

Formulations suitable for intravenous administration such as injection or infusion formulation, comprise sterile isotonic solutions of levosimendan or its active metabolite (II) and vehicle, preferably aqueous solutions. Typically an intravenous infusion solution comprises from about 0.01 to 0.1 mg/ml of levosimendan or its active metabolite (II).

Salts of levosimendan or its active metabolite (II) may be prepared by known methods. Pharmaceutically acceptable salts are useful as active medicaments, however, preferred salts are the salts with alkali or alkaline earth metals.

### PHARMACEUTICAL EXAMPLES

### Example 1

### Oral Capsule

Hard gelatin capsule size 3

| | |
|---|---|
| Levosimendan | 2.0 mg |
| Lactose | 198 mg |

The pharmaceutical preparation in the form of a capsule was prepared by mixing levosimendan with lactose and placing the powdery mixture in hard gelatin capsule.

### Example 2

### Concentrate Solution for Intravenous Infusion

| | |
|---|---|
| (a) levosimendan | 2.5 mg/ml |
| (b) Kollidon PF12 | 10 mg/ml |
| (c) citric acid | 2 mg/ml |
| (d) dehydrated ethanol | ad 1 ml (785 mg) |

The concentrate solution was prepared by dissolving citric acid, Kollidon PF121 and levosimendan to dehydrated ethanol in the sterilized preparation vessel under stirring. The resulting bulk solution was filtered through a sterile filter (0.22 μm). The sterile filtered bulk solution was then aseptically filled into 8 ml and 10 ml injection vials (with 5 ml and 10 ml filling volumes) and closed with rubber closures.

The concentrate solution for intravenous infusion is diluted with an aqueous vehicle before use. Typically the concentrate solution is diluted with aqueous isotonic vehicles, such as 5% glucose solution or 0.9% NaCl solution so as to obtain an aqueous intravenous solution, wherein the amount of levosimendan is generally within the range of about 0.001-1.0 mg/ml, preferably about 0.01-0.1 mg/ml.

Experiments

Experiment 1. Heart weight/body weight ratio, SERCA2/NCX protein ratio and atrial natriuretic peptide (ANP) mRNA expression

Methods

6-week-old male Dahl salt-sensitive rats (SS/JrHsd) received the following diet and drug regimens for 7 weeks: 1) Dahl SS controls on high salt diet, 2) Dahl SS rats on high salt diet+high-dose levosimendan (10 mg/l of levosimendan in drinking water), 3) Dahl SS rats on high salt diet+low-dose levosimendan (1 mg/l of levosimendan in drinking water) and 4) Dahl SS controls on low salt diet. High salt diet was produced by adding NaCl to commercial low salt diet. The consumption of drinking water and food as well as the body weight and general health of the animals were monitored. Systolic blood pressure was measured by using a tail cuff blood pressure at week 3.5 and week 7. At the end the study the hearts was excised, washed with ice-cold saline, blotted dry and weighed.

Myocardial SERCA2 and NCX expressions were determined by Western blot analysis using standard procedure. Myocardial samples were homogenized in extraction buffer and protease inhibitor. Myocardial samples (15 μg protein per lane) were electrophoretically separated by SDS-PAGE (8% Acryl amide). The proteins were transferred to a PVDF membrane by semi-dry blotting in electrophoresis device. After transfer the membrane was blocked in +4° C. in 5% milk powder-TBS-0.01% Tween solution. The membrane was washed and probed for 1 h at room temperature with the primary antibody (rabbit anti-NCX, 1:5000 AD). After washing, the membrane was probed with peroxidase-conjugated secondary antibody (anti-rabbit 1:5000; Chemicon). Detection was accomplished with an enhanced chemiluminescence kit and the blots were exposed to x-ray film. The membrane was stripped from antibodies and after washing it was re-probed with a second antibody (rabbit anti-Serca2, 1:5000 Abcam), probing with secondary antibody and detection were done as described above. The films were scanned in a densitometer and a semi-quantitative measurement of the relative intensity of each protein band was performed using the “GeneSnap”-software program.

Total RNA from the rat hearts were collected, treated with DNAse 1 and reverse transcribed to cDNA by incubation of 50 min in 45° C. with presence of reverse transcription enzyme (Enhanced avian HS RT-PCR kit, Sigma Chemicals Co.). 1 μl of cDNA was subjected to a quantitative real time polymerase chain reaction by Lightcycler instrument (Roche Diagnostics) for detection of ANP and GAPDH mRNAs. GAPDH served as housekeeping gene. The samples were amplified by using FastStart DNA Master SYBR Green 1 (Roche Diagnostics) in presence of 0.5 μM of following primers: ANP forward CCGATAGATTCTGCCCTCTTGAA, reverse CCCGAAGCAGCTTGATCTTC; GAPDH forward GGATGCAGGGATGATGTTCT, reverse GAAGGGCTCATGACCACAGT. The PCR amplifications consisted of 10 minutes incubation in 95° C. following 30 cycles of 15 seconds in 95° C., annealing for 5 seconds in 62° C. and 10 seconds in 72° C. for ANP; 10 minutes incubation in 95° C. following 35 cycles of 15 seconds in 95° C., annealing for 5 seconds in 55° C. and 10 seconds in 72° C. for GAPDH. After amplification the quality of PCR products were analyzed with the melting step consisting of heating to 95° C., cooling to annealing temperature for 15 seconds, and finally a slow rise in temperature to 95° C. with a continuous acquisition of fluorescence decline. The quantity of ANP and GAPDH PCR products were quantified with an external standard curve amplified from purified PCR product.

Results

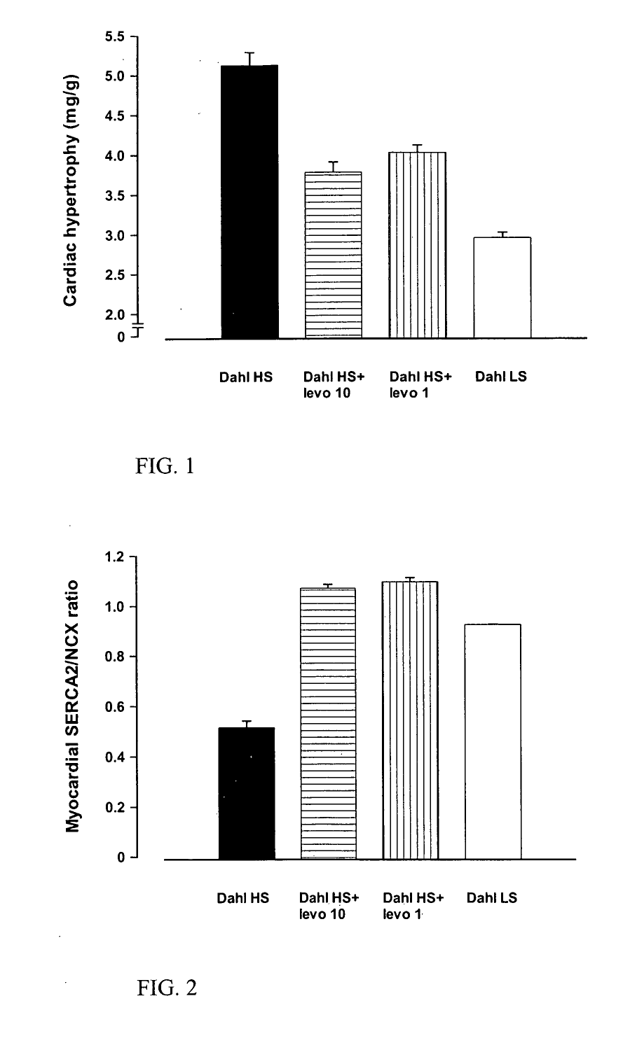

FIG. 1 shows the effect of levosimendan on the ratio of heart weight to body weight. Dahl SS rats on high salt diet developed pronounced hypertension with cardiac hypertrophy. Both levosimendan doses equally prevented the development of cardiac hypertrophy when measured as heart weight-to-body weight-ratio.

High-dose levosimendan produced a transient decrease in blood pressure, whereas low-dose levosimendan did not influence blood pressure in Dahl DD rats (data not shown). Thus, changes in blood pressure do not explain the beneficial effect of levosimendan in cardiac hypertrophy.

As shown in FIG. 2, in Dahl SS rats on high salt diet the myocardial SERCA2-to-NCX-ratio decreased as compared to Dahl SS controls on low salt diet indicating diastolic dysfunction. Both levosimendan doses increased SERCA2-to-NCX-ratio in the heart thus indicating improvement in diastolic function.

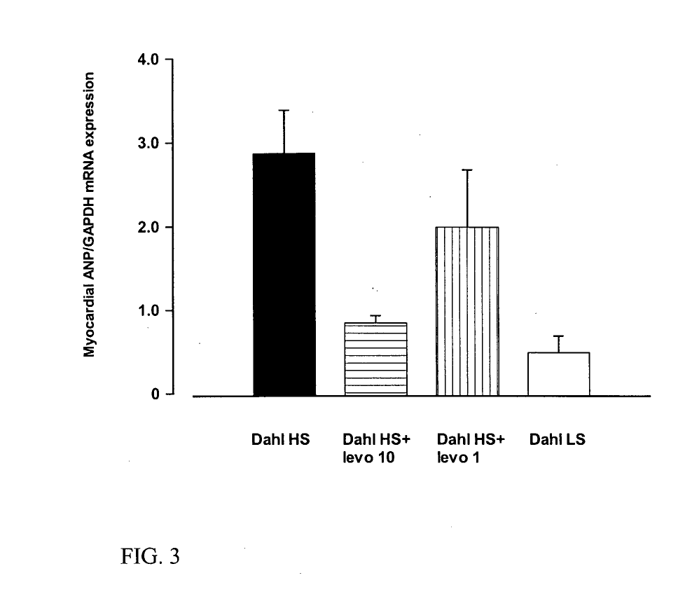

Increased expression of atrial natriuretic peptide (ANP) in cardiac tissue has been used as a biomarker for the development of cardiac hypertrophy. As shown in FIG. 3, myocardial ANP mRNA expression was increased by five-fold in rats on high salt diet. High dose levosimendan was able to decrease ANP mRNA expression to levels found in low salt diet controls.

Experiment 2. Echocardiography

Methods

6-week-old male Dahl salt-sensitive rats (SS/JrHsd) received the following diet and drug regimens: Dahl salt-sensitive rats on low salt diet (1), on high salt diet (2), on high salt diet treated with 10 mg/l of levosimendan in drinking water (3), on high salt diet treated with 1 mg/l of levosimendan in drinking water (4) and on high salt diet treated with 0.5 mg/kg of the active metabolite (II) (OR-1896) of levosimendan in drinking water (5). High salt diet was produced by adding NaCl to commercial low salt diet. After 3.5 weeks transthoracic echocardiography was performed using a Toshiba Ultrasound System and a 15 MHz linear transducer under light isoflurane anesthesia. Using two-dimensional imaging, a short axis view of the left ventricle at the level of the papillary muscles was obtained and the two-dimensionally guided M-mode recording through the anterior and posterior walls of the left ventricle was obtained.

Results

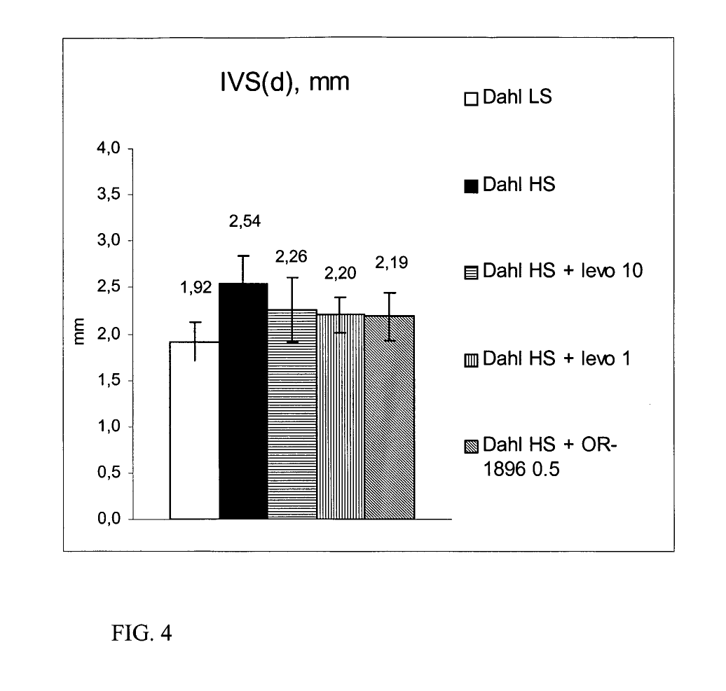

Interventricular septum (IVS) wall thickness (mm) of the heart as measured from the M-mode tracings is shown in FIG. 4 for animal groups 1-5 described above. Increased heart wall thickness due to hypertrophy can be seen in the high salt diet group as compared to low salt diet group. Levosimendan and its active metabolite (II) were able to significantly reduce the increased heart wall thickness of the high salt diet group.

1-3. (canceled)

4. A method for the treatment or prevention of cardiac hypertrophy in a mammal, said method comprising administering to a mammal in need thereof an effective amount of levosimendan or its metabolite (II):
(R)-N-[4-(1,4,5,6-tetrahydro-4-methyl-6-oxo-3-pyridazinyl)phenyl]acetamide (II) or a pharmaceutically acceptable salt of levosimendan or its metabolite (II).

5. A method according to claim 4, wherein the cardiac hypertrophy is hypertension-induced cardiac hypertrophy.

6. A method for treatment or prevention of diastolic heart failure resulting from cardiac hypertrophy, said method comprising administering to a mammal in need thereof an effective amount of levosimendan or its metabolite (II):
(R)-N-[4-(1,4,5,6-tetrahydro-4-methyl-6-oxo-3-pyridazinyl)phenyl]acetamide (II) or a pharmaceutically acceptable salt of levosimendan or its metabolite (II).
